(19) United States
(12) Patent Application Publication
(10) Pub. No.: US 2023/0157586 A1
(43) Pub. Date: May 25, 2023

(54) FOUR-COMPARTMENT DIFFUSION MODEL OF CORTISOL IN HUMANS

(71) Applicants: United States Government as Represented by the Department of Veterans Affairs, Washington, DC (US); The Florida International University Board of Trustees, Miami, FL (US)

(72) Inventors: Richard I. Dorin, Albuquerque, NM (US); Clifford R. Qualls, Mesa, AZ (US); Frank K. Urban, Palmetto Bay, FL (US)

(21) Appl. No.: 17/993,282

(22) Filed: Nov. 23, 2022

Related U.S. Application Data
(60) Provisional application No. 63/283,107, filed on Nov. 24, 2021.

(51) Int. Cl. A61B 5/145; G16H 40/67

(52) U.S. Cl. CPC A61B 5/145; G16H 40/67

(57) ABSTRACT

Methods, systems, and apparatuses for modeling and determining of the cortisol elimination rate in patients are described. One or more physiological measurements associated with a patient may be determined and applied to a multi-compartment model. One or more physiological parameters, including the cortisol elimination rate, may be determined based on applying the one or more physiological measurements to the multi-compartment model.

### CROSS REFERENCE TO RELATED PATENT APPLICATION

This Application claims priority to U.S. Provisional Application No. 63/283,107, filed Nov. 24, 2021, which is herein incorporated by reference in its entirety.

### BACKGROUND

Cortisol secretion rate (CSR) is an important measure of adrenocortical function. The CSR cannot be measured directly and so a method for determining it from measurable parameters is required. Typically these parameters include the time variation of the concentration of total cortisol and may also include the time variation of the concentration of free cortisol. Rates of cortisol appearance during critical illness have been estimated using numerical methods. For example, numerical methods use models to represent the physiology of cortisol disposition, and the models are typically formulated mathematically as differential equations. Given the measurements and model, numerical methods may be used to solve these equations to calculate CSR assuming other parameters in the model are known or can be computed simultaneously. However, there is uncertainty about which of the models in current use, if any, are sufficiently accurate for clinical application.

A number of models for cortisol concentration as a function of time have been previously developed. Three alternative models of cortisol disposition (Models 1-3) have been applied to human subjects in the past decade. Model 1 considered the total cortisol (measured in the plasma-concentration space) as a single compartment. Model 2 considered free cortisol (measured in the plasma-concentration space) as a single compartment. Model 3 considered the dynamic equilibrium of cortisol among three compartments measured in the plasma-concentration space, namely free, corticosteroid binding globulin (CBG)-bound and albumin-bound cortisol, according to principles of mass action.

In previous literature, significant correlations between CBG concentration and cortisol half-life were observed using Models 1 and 2. These associations have suggested that CBG may exert a measurable, physiologically meaningful effect on the rate of cortisol elimination. An alternative interpretation is that the relationship between cortisol half-life and CBG concentration (or cortisol binding affinity) may be due to a modeling error. In addition, the two previous cortisol concentration models did not take binding of cortisol to blood proteins into account. Although the 3-compartment model met with success under restricted experimental conditions, it did not allow fully fitting of certain data. Essentially, the estimates of cortisol elimination rates varied according to the model used. Although the multi-compartment Model 3 was superior to the single-compartment Models 1-2, all three models demonstrated unsatisfactory performance characteristics.

### SUMMARY

It is to be understood that both the following general description and the following detailed description are exemplary and explanatory only and are not restrictive.

In an embodiment, disclosed are methods comprising determining one or more physiological measurements associated with a patient, applying the one or more physiological measurements to a multi-compartment model, wherein the multi-compartment model represents a bidirectional flux by diffusion of a hormone between a vascular compartment and an extravascular compartment of the patient, determining, based on the application of the one or more physiological measurements to the multi-compartment model, one or more physiological parameters associated with the patient, and determining, based on the one or more physiological parameters, one or more medical conditions associated with the patient.

In an embodiment, disclosed are methods comprising determining one or more physiological measurements associated with a patient, applying the one or more physiological measurements to a multi-compartment model, wherein the multi-compartment model represents a bidirectional flux by diffusion of a hormone between a vascular compartment and an extravascular compartment of the patient, determining, based on the application of the one or more physiological measurements to the multi-compartment model, one or more physiological parameters associated with the patient, and administering, based on the one or more physiological parameters, a treatment of one or more medical conditions associated with the patient.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example system for determining a cortisol elimination rate;

FIG. 2 shows a flowchart of an example solution algorithm;

FIGS. 3A-3D show example measurement data and results for the model equations;

FIG. 4 shows a flowchart of an example method;

FIG. 5 shows a flowchart of an example method;

FIG. 6 shows a block diagram of an example system and computing device for determining a cortisol elimination rate.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

As used herein the terms “patient,” “subject,” or “person” may indicate a person associated with the determination of one or more physiological parameters, such as a hormone elimination rate, an extravascular volume fraction, and a permeability constant related to diffusion.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, the methods and systems may take the form of a computer program product on a computer-readable storage medium (e.g., non-transitory) having processor-executable instructions (e.g., computer software) embodied in the storage medium. More particularly, the present methods and systems may take the form of web-implemented computer software. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks. In addition, some of these functions may be carried out using complex programmable logic devices (CPLDs) or other programmable logic devices.

The processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks. In addition, some of these functions may be carried out using logic devices which do not operate by sequential operations of programmed steps.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions and logic circuitry.

Methods and systems are described for determining one or more physiological parameters of a human subject, such as a cortisol elimination rate, an extravascular volume fraction, and a permeability constant associated with diffusion. The physiological parameters are important for predicting one or more physiological attributes associated with a patient, such as time-varying concentrations of plasma free cortisol in a vascular volume, CBG-bound cortisol in a vascular volume, albumin-bound cortisol in a vascular volume, and free cortisol in an extravascular volume. For example, one or more physiological measurements associated with a patient may be determined. The one or more physiological measurements may comprise one or more of a time series of blood cortisol concentrations, plasma concentrations associated with corticosteroid binding globulin (CBG) and cortisol, a total CBG concentration, a total albumin concentration, a blood volume, a plasma volume, vascular volume, or extravascular volume. For example, the one or more physiological measurements may be applied to a multi-compartment. The multi-compartment model may represent a bidirectional flux by diffusion of a hormone between a vascular compartment and an extravascular compartment. The hormone may comprise one or more of free cortisol, testosterone, aldosterone, vitamin D, or a peptide hormone. The multi-compartment model may comprise four non-linear differential equations. The four non-linear differential equations may represent time-varying concentrations of plasma free cortisol in the vascular volume, CBG-bound cortisol in the vascular volume, albumin-bound cortisol in the vascular volume, and free cortisol in the extravascular volume. The one or more physiological parameters may be determined based on the application of the one or more physiological measurements to the multi-compartment model. One or more medical conditions associated with the patient may be determined based on the one or more physiological parameters. In addition, a treatment may be administered based on the one or more medical conditions.

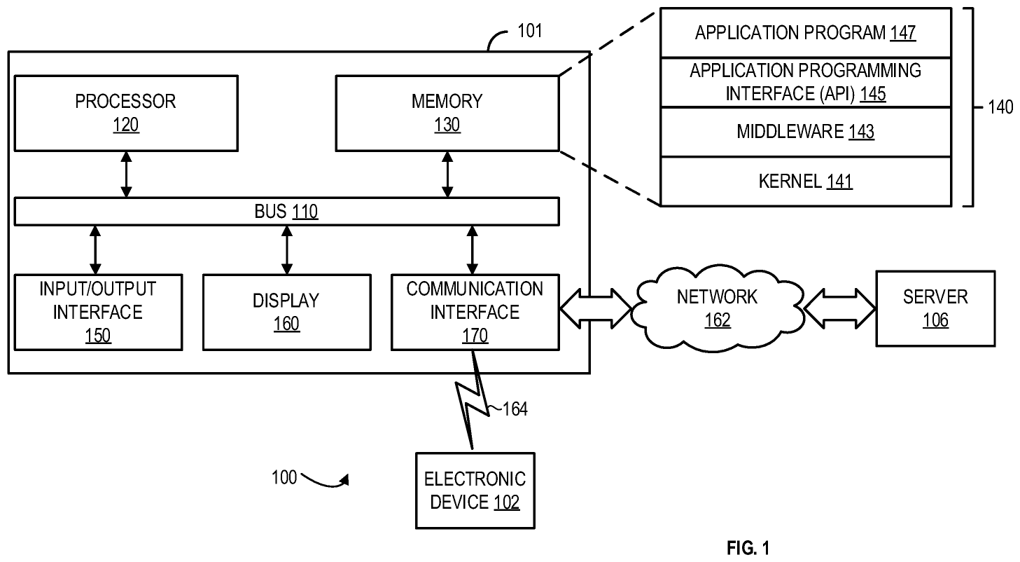

FIG. 1 shows an example system 100 for determining one or more physiological parameters of a human subject (e.g., patient), such as a cortisol elimination rate, an extravascular volume fraction, and a permeability constant associated with diffusion. The system 100 may be configured to process one or more physiological measurements associated with a patient and apply the physiological measurements to a multi-compartment model to determine the one or more physiological parameters associated with the patient. Referring to FIG. 1, the system 100 may comprises a device 101. The device 101 may include a bus 110, one or more processors 120, a memory 130, an input/output interface 150, a display 160, and a communication interface 170. In an example, the device 101 may omit at least one of the aforementioned constitutional elements or may additionally include other constitutional elements. The device 101 may comprise, for example, a mobile phone, a smart phone, a tablet computer, a laptop, a desktop computer, a smartwatch, a smart glass, and the like.

The bus 110 may include a circuit for connecting the bus 110, the one or more processors 120, the memory 130, the input/output interface 150, the display 160, and/or the communication interface 170 to each other and for delivering communication (e.g., a control message and/or data) between the bus 110, the one or more processors 120, the memory 130, the input/output interface 150, the display 160, and/or the communication interface 170.

The one or more processors 120 may include one or more of a Central Processing Unit (CPU), an Application Processor (AP), and a Communication Processor (CP). The one or more processors 120 may control, for example, at least one of the bus 110, the one or more processors 120, the memory 130, the input/output interface 150, the display 160, and/or the communication interface 170 and/or may execute an arithmetic operation or data processing for communication. For example, the one or more processors 120 may be configured to determine the one or more physiological parameters based on the one or more physiological measurements and determine one or more medical conditions associated with the patient based on the one or more physiological parameters. The processing (or controlling) operation of the one or more processors 120 according to various embodiments is described in detail with reference to the following drawings.

The processor-executable instructions executed by the one or more processor 120 may be stored and/or maintained by the memory 130. The memory 130 may include a volatile and/or non-volatile memory. The memory 130 may store, for example, a command or data related to at least one different constitutional element of the electronic device 101. According to various exemplary embodiments, the memory 130 may store a software and/or a program 140. The program 140 may include, for example, a kernel 141, a middleware 143, an Application Programming Interface (API) 145, and/or an application program (or an “application”) 147, or the like, configured for controlling one or more functions of the device 101 and/or an external device. At least one part of the kernel 141, middleware 143, or API 145 may be referred to as an Operating System (OS). The memory 130 may include a computer-readable recording medium having a program recorded therein to perform the method according to various embodiments by the processor 120.

The kernel 141 may control or manage, for example, system resources (e.g., the bus 110, the processor 120, the memory 130, etc.) used to execute an operation or function implemented in other programs (e.g., the middleware 143, the API 145, or the application program 147). Further, the kernel 141 may provide an interface capable of controlling or managing the system resources by accessing individual constitutional elements of the device 101 in the middleware 143, the API 145, or the application program 147.

The middleware 143 may perform, for example, a mediation role so that the API 145 or the application program 147 can communicate with the kernel 141 to exchange data. Further, the middleware 143 may handle one or more task requests received from the application program 147 according to a priority. For example, the middleware 143 may assign a priority of using the system resources (e.g., the bus 110, the processor 120, or the memory 130) of the device 101 to at least one of the application programs 147. For instance, the middleware 143 may process the one or more task requests according to the priority assigned to the at least one of the application programs, and thus may perform scheduling or load balancing on the one or more task requests.

The API 145 may include at least one interface or function (e.g., instruction), for example, for file control, window control, video processing, or character control, as an interface capable of controlling a function provided by the application 147 in the kernel 141 or the middleware 143.

For example, the input/output interface 150 may play a role of an interface for delivering an instruction or data input from a user or a different external device(s) to the different constitutional elements of the device 101. Further, the input/output interface 150 may output an instruction or data received from the different constitutional element(s) of the electronic device 101 to the different external device (e.g., electronic device 102, server 106, etc.).

The display 160 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 160 may display, for example, a variety of contents (e.g., text, image, video, icon, symbol, etc.) to the user. The display 160 may include a touch screen. For example, the display 160 may receive a touch, gesture, proximity, or hovering input by using a stylus pen or a part of a user's body.

The communication interface 170 may establish, for example, communication between the device 101 and an external device (e.g., an electronic device 102 or a server 106). For example, the communication interface 170 may communicate with the external device (e.g., the electronic device 102 or the server 106) by being connected to a network 162 through wireless communication or wired communication.

For example, as a cellular communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. Further, the wireless communication may include, for example, a near-distance communication 164. The near-distance communications 164 may include, for example, at least one of Wireless Fidelity (WiFi), Bluetooth, Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like. The network 162 may include, for example, at least one of a telecommunications network, a computer network (e.g., LAN or WAN), the internet, and a telephone network.

The server 106 may include a group of one or more servers. As an example, all or some of the operations executed by the device 101 may be executed in a different one or a plurality of electronic devices (e.g., the electronic device 102 or the server 106). In an example, if the device 101 needs to perform a certain function or service either automatically or based on a request, the device 101 may request at least some parts of functions related thereto alternatively or additionally to a different electronic device (e.g., the electronic device 102 or the server 106) instead of executing the function or the service autonomously. The different electronic devices (e.g., the electronic device 102 or the server 106) may execute the requested function or additional function, and may deliver a result thereof to the device 101. As an example, the electronic device 102 may comprise one or more complex programmable logic devices (CPLDs) or other programmable logic devices. The electronic device 102 may be configured to process the one or more physiological measurements to determine the one or more physiological parameters. For example, the electronic device 102 may be configured to receive the one or more physiological measurements from the device 101, wherein the electronic device 102 may process the one or more physiological measurements to determine the one or more physiological parameters. For example, the electronic device 102 may determine the one or more physiological parameters based on applying the one or more physiological measurements to a multi-compartment model. The electronic device 102 may send the one or more physiological parameters to the device 101, wherein the device 101 may determine one or more medical conditions based on the one or more physiological parameters and provide, or cause, a treatment associated with the one or more medical conditions. As a further example, the device 101 may send the determined one or more physiological parameters to the electronic device 102, wherein the electronic device 102 may be configured to determine one or more medical conditions based on the one or more physiological parameters and send a treatment recommendation associated with the one or more medical conditions to the device 102. As a further example, the server 106 may be configured to receive the one or more physiological measurements from the device 101 and process the one or more physiological measurements to determine the one or more physiological parameters. As a further example, the device 101 may send the determined one or more physiological parameters to the server 106, wherein the server 106 may be configured to determine one or more medical conditions based on the one or more physiological parameters and send a recommendation of a treatment associated with the one or more medical conditions to the device 101. The device 101 may provide the requested function or service either directly or by additionally processing the received result. For example, a cloud computing, distributed computing, or client-server computing technique may be used.

Each of the constitutional elements described in the present document may consist of one or more components, and names thereof may vary depending on a type of an electronic device. The device 101 may include at least one of the constitutional elements described in the present document. Some of the constitutional elements may be omitted, or additional other constitutional elements may be further included. Further, some of the constitutional elements of the device 101 according to various exemplary embodiments may be combined and constructed as one entity, so as to equally perform functions of corresponding constitutional elements before combination.

As a further example, one or more devices (e.g., device 101, electronic device 102, or server 106) may be configured to determine the one or more physiological parameters associated with the patient, such as a cortisol elimination rate (α), an extravascular volume fraction (V_{f}), and a permeability constant associated with diffusion (k). The physiological parameters are important for predicting one or more physiological attributes associated with the patient, such as time-varying concentrations of plasma free cortisol in a vascular volume, CBG-bound cortisol in a vascular volume, albumin-bound cortisol in a vascular volume, and free cortisol in an extravascular volume. For example, the one or more devices may determine one or more physiological measurements associated with a patient. The one or more physiological measurements may comprise one or more of a time series of blood cortisol concentrations, plasma concentrations associated with corticosteroid binding globulin (CBG) and cortisol, a total CBG concentration, a total albumin concentration, a blood volume, a plasma volume, vascular volume, or extravascular volume. The one or more physiological measurements may be applied to a multi-compartment. The multi-compartment model may represent a bidirectional flux by diffusion of a hormone between a vascular compartment and an extravascular compartment. For example, the multi-compartment model may comprise four non-linear differential equations. The hormone may comprise one or more of free cortisol, testosterone, aldosterone, vitamin D, or a peptide hormone. The four non-linear differential equations may represent time-varying concentrations of plasma free cortisol in a vascular volume, CBG-bound cortisol in a vascular volume, albumin-bound cortisol in a vascular volume, and free cortisol in an extravascular volume.

The four non-linear differential equations may be expressed as:

$$\frac{{dX}_{F}}{dt} = - \{ α + {κ}_{1}^{C} ( {X}_{TotCBG} - {X}_{C} ) + {κ}_{1}^{A} ( {X}_{TotA} - {X}_{A} ) \} * {X}_{F} + {κ}_{- 1}^{C} * {X}_{C} + {κ}_{- 1}^{A} * {X}_{A} - \frac{k}{V} * ( {X}_{F} - {X}_{Fe} ) + {X}_{F} ; \qquad 1 )$$

$$\frac{{dX}_{C}}{dt} = - {κ}_{1}^{C} * {X}_{C} + {κ}_{1}^{C} ( {X}_{TotCBG} - {X}_{C} ) * {X}_{F} ; \qquad 2 )$$

$$\frac{{dX}_{A}}{dt} = - {κ}_{- 1}^{A} * {X}_{A} + {κ}_{1}^{A} ; and \qquad 3 )$$

$$\frac{{dX}_{Fe}}{dt} = + \frac{k}{Ve} * ( {X}_{F} - {X}_{Fe} ) ; \qquad 4 )$$

where,

$$\frac{dX}{dt}$$

is a derivative of a concentration X with respect to time t, change in concentration per unit time, or rate of change of X;
X_{F} is the time-varying concentration of plasma free cortisol;
X_{Fe} is the time-varying concentration of free cortisol in extravascular volume (Ve);
V is vascular volume (plasma);
X_{C} is the time-varying concentration of Corticosteroid-Binding Globulin (CBG) bound cortisol;
X_{A} is the time-varying concentration of albumin-bound cortisol;
X_{TotCBG} is a total CBG concentration;
X_{TotA} is a total albumin concentration;
Z_{F} is a time-varying free cortisol appearance rate;
α is an elimination rate constant for free cortisol;
k is a permeability constant in Fick's first law,

$$\frac{{dQ}_{Fe}}{dt} = k ( {X}_{F} - {X}_{Fe} ) ,$$

where Q_{Fe} is a quantity of free cortisol in an extravascular compartment;
κ₁^{C} is an association rate constant (on-rate);
κ₋₁^{C} is a dissociation rate constant (off-rate);
κ₁^{A} is an association rate-constant (on-rate);
κ₋₁^{A} is a dissociation rate constant (off-rate); and
X_{TotF} is the time-varying total cortisol concentration in the vascular volume (V) and equals the sum of free, CBG-bound, and albumin-bound cortisol concentrations in V (X_{TotF}=X_{F}+X_{C}+X_{A}), here measured.

Equation (1) is derived from conservation of mass for cortisol molecules and has an additional term to account for the rate of diffusion of free cortisol between the vascular volume and extravascular volume. Equations (2) and (3) represent the standard first order rate equations for binding and unbinding. Equation (4) is Fick's first law of diffusion algebraically manipulated to be expressed in terms of concentration rate rather than flux, Q. Equation (4) accounts for a rate of diffusion of free cortisol between the vascular volume and the extravascular volume. The subtractive factors are the elimination rate, rate of association of free cortisol to CBG and albumin (also shown in equations (2) and (3)), and rate of diffusion of free cortisol into the extravascular volume. The additive factors include secretion rate, rate of disassociation from CBG and albumin (as also shown in equations (2) and (3)), and diffusion rate out of the extravascular volume.

The model is solved for the elimination rate constant (α), the diffusion constant and geometry (k), the volume fraction (V_{fr}) where V_{fr}=Ve/Vb in which Vb is body volume estimated as body weight/density of water. As an example, the multiplication of the concentrations by associated volumes would allow expression in terms of mass in moles.

Serum concentrations of CBG and total free cortisol in CBG-deficient subjects homozygous for GLy237Val substitution in the CBG (SERPINA6) gene as well as in vitro cortisol binding studies of the mutant CBG protein have been previously reported. CBG-deficient subjects have abnormally low CBG concentrations (X_{TotCBG}) by definition and, compared to wild type CBG protein, the mutant CBG protein has markedly decreased cortisol binding affinity (higher equilibrium dissociation constant K_{D}). The computation of K_{D} for CBG-deficient subjects is needed for the analyses given below. The computation of K_{D} for cortisol and CBG binding is based on multiple, paired measurements of free cortisol concentrations (X_{F}) and total cortisol concentration (X_{TotF}) and their equilibrium relationship. The equilibrium can be seen by setting the left hand side of equations 2) and 3) equal to zero; these equilibria take place in considerably less than a second. All measurements of X_{F} and X_{TotF} in test tubes are under these equilibrium conditions. These conditions yield

$${X}_{A} = \frac{{X}_{TotA} * {X}_{F}}{{κ}_{A} + {X}_{F}} and {X}_{C} = \frac{{X}_{TotCBG} * {X}_{F}}{{κ}_{C} + {X}_{F}} ,$$

where

$${K}_{A} = \frac{{κ}_{- 1}^{A}}{{κ}_{1}^{A}} and {K}_{C} = \frac{{κ}_{- 1}^{C}}{{κ}_{1}^{C}}$$

are the equilibrium dissociation constants.

Thus, X_{F} is obtained from X_{TotF}=X_{F}+X_{C}+X_{A} as a cubic equation solution and is equal to a factor* X_{TotF}, where the factor is a known expression in X_{TotA}, K_{A}, X_{TotCBG}, and K_{C}. Here, X_{TotCBG} is measured, and K_{C} and K_{A} may be estimated simultaneously by nonlinear least squares methods using 15×2 points of measured pairs (X_{F}, X_{TotF}) for CBG-deficient subjects. Finally, X_{TotF} is the time-varying total cortisol concentration in the vascular volume (V) and equals the sum of free, CBG-bound, and albumin-bound cortisol concentrations in V (X_{TotF}=X_{F}+X_{C}+X_{A}).

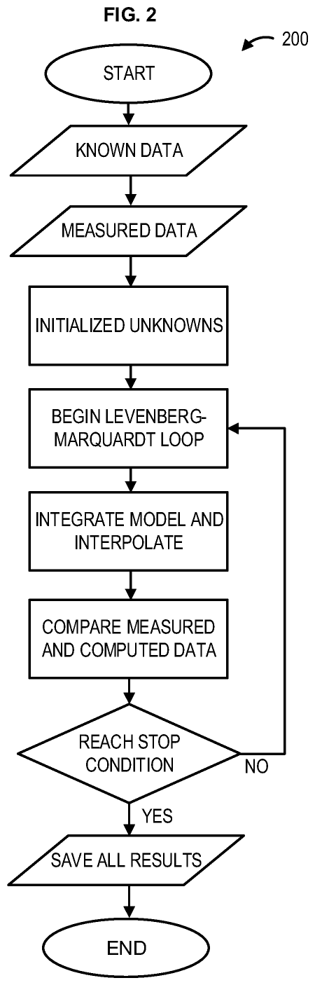

FIG. 2 shows a flowchart of an example solution algorithm 200. FIG. 2 shows the sequence beginning with data input followed by the Levenberg-Marquardt loop which contains the numerical integration of the model. The loop continues until the stopping conditions are reached. The model defined by the above multi-compartment equations cannot be inverted directly to provide solutions from the measurements. The measured data consists of a time series of blood cortisol concentrations up to 480 minutes at intervals of 10 minutes or more across the time span. FIG. 2 shows the flow diagram for the solving algorithm. Due to the nature of numerical methods, an exact match between the two methods is not expected. It can be seen that the overall solution process is the Levenberg Marquardt least-squares method for solving nonlinear equations in an iterative loop. Within the loop is a numerical integration process used to solve the model equations. Solutions were obtained using weighted least squares, where weights are 1/SD², wherein SD refers to the instrumental measurement accuracy.

The first step in the solution is to initialize the Levenberg-Marquardt Solver. This initialization begins with the identification of the unknowns. The next step includes selecting stopping conditions and establishing any options which might be used during the solution process. Although, the Matlab “lsqnonlin” function was used, other functions may be used including purpose-written code. Upper and lower bounds are specified for each of the three solved parameters.

It is necessary to evaluate the model functions within each loop. A variable-step is used, such as the variable-order (VSVO) solver based on the numerical differentiation formulas (NDFs) of orders 1 to 5. Although the Matlab ODE15s solver was selected, others may be used as well. For the initial integration, values for all of the parameters must be employed, including those which are sought in solution.

Thus, the procedure requires choosing initial estimates for each of the unknowns, wherein these estimates are adjusted through each iteration to obtain numerical solutions by the Levenberg-Marquardt algorithm. Standard initialization and options were used. The integration produces a large number of solutions at more than 1,000 time points from which solutions at the measurement time points are obtained by interpolation. Thus, the algorithm solves numerically for three parameters: α (elimination rate constant), V_{fr} (effective volume fraction), and the k (derived diffusion factor).

The stopping conditions for the integration numerical method comprise a relative tolerance (e.g., 3×10⁻¹⁴) and absolute tolerance (e.g., 1×10⁻¹⁵) for each parameter. Relative tolerance is the allowable error tolerance at each integration step relative to the state vector and absolute tolerance specifies the maximum allowable tolerance for each step for each parameter in the solution. The stopping conditions for the Levenberg-Marquardt numerical method comprise the tolerance function (e.g., 1×10⁻²⁴), a tolerance in X (e.g., 1×10⁻²⁴), maximum function evaluations (e.g., 1000), and max iterations (e.g., 40). Both of the tolerance functions are thus set to default to the smallest number which can be represented on the computer. Neither the Levenberg-Marquardt nor the integration algorithms have been observed to be sensitive to these conditions. To avoid divergence to unrealistic local minima, boundaries may be set to find solutions in the ranges of 0<α<0.1, 0<k<0.1, and 0.95<Vfr<1.05.

In one example, particular values for 37° C. used in equations (1) through (4) are the same for each subject. These include κ₁^{A}, κ₋₁^{A}, κ₁^{C}, and κ₋₁^{C}. Individually measured plasma concentrations include CBG and cortisol. Instead of measuring the albumin concentration, X_{TotA}, a population average may be used. Of the remaining parameters, X_{TotCBG} is measured for each subject. Values for the remaining concentrations are determined in the solution including the three unknowns, elimination rate (α), diffusion constant (k), and volume fraction (V_{fr}).

Initial conditions may be determined by dividing the mass of cortisol administered (20 mg=55,249 nanomole) by the vascular volume (V), which was considered to be the plasma volume. Blood volumes were calculated according to weight, height, and gender using Nadler's formula. Plasma volumes were obtained by multiplying blood volume by the plasma fraction (100-hematocrit)/100. Mean population hematocrit values of 0.45 may be used for men and 0.40 may be used for women. In one example, the following table shows an example set of numbers and units that apply to one of the subjects. Of these, the value of CBG is measured for each subject.

TABLE I

| subject | value | units |
|---|---|---|
| κ₁^{A} | 8.95 × 10³ | (M*s)⁻¹ |
| κ₋₁^{A} | 2.2 | s⁻¹ |
| κ₁^{C} | 3.62 × 10⁷ | (M*s)⁻¹ |
| κ₋₁^{C} | 0.88 | s⁻¹ |
| X_{TotCBG} | 846.6 | nanomolar |
| X_{TotA} | 647,150 | nanomolar |

In this example, the demographics, subject characteristics, assay procedures, experimental protocol, and measured data have been previously reported. Endogenous cortisol secretion was suppressed by administration of DEX (4 mg) at 2200 h on the evening prior to hydrocortisone administration at 0900 h. The data used for model comparisons included X_{TotCBG} and serial total plasma cortisol concentrations, X_{TotF}, measured at intervals ranging from 10-480 min following administration of an iv hydrocortisone bolus (20 mg) in two clinical groups: healthy volunteer (HV, n=6) and CBG deficient (n=2). Albumin concentrations were not measured in the provided data. Thus, substitute albumin concentrations are computed, based on median age and gender-stratified data.

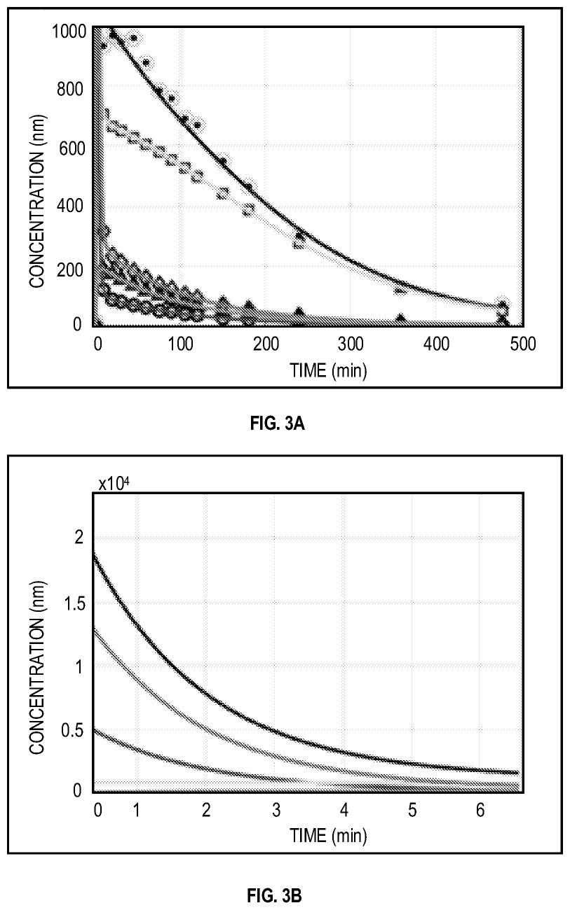

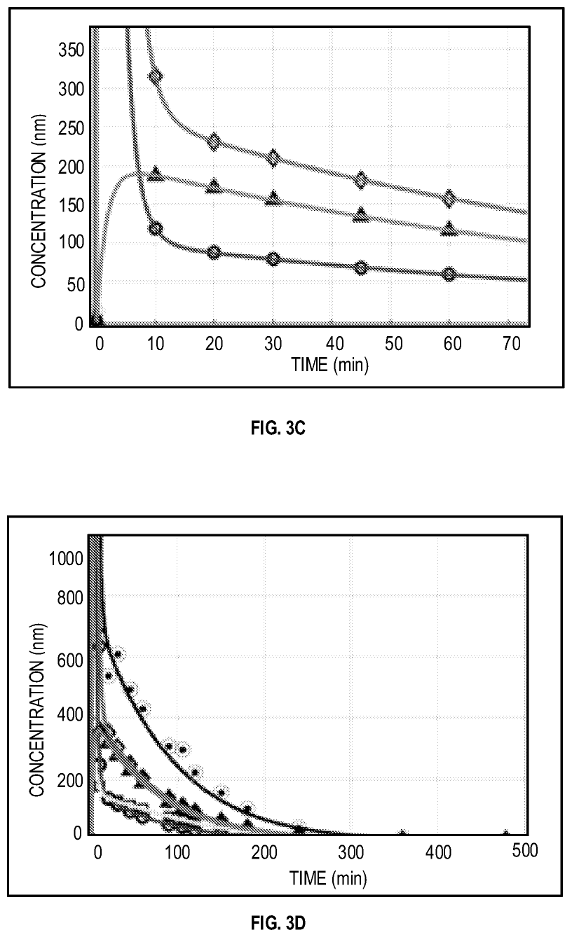

FIGS. 3A-3D show the measured data and results for the model equations. FIG. 3A shows a graph representing the measured data and solutions for the model equations for a representative subject. In FIG. 3A, the circled dots represent the measured data of total cortisol concentration taken in a time sequence. The solid lines are obtained based on the numerical method described above. Based on the graph, the solution for total vascular cortisol, the uppermost curve, is a fit to the measured data which is confirmed by the residuals. The next curve down is a plot of vascular CBG-bound cortisol (□) followed by vascular albumin-bound cortisol (⋄). The next curve down is the concentration of free cortisol in the extravascular volume (▴) and the lowest curve beyond 10 minutes is the concentration of vascular free cortisol (∘).

As shown in Table I, the association (on) rate of CBG, κ₁^{C}, is 4045 times greater than κ₁^{A} for albumin. At the same time the concentration of CBG, X_{TotCBG}, is 764 times less than that of albumin, X_{TotA}. This shows why there is about 5 times the vascular concentration of CBG-bound cortisol, X_{C}, than albumin-bound, X_{A}. The much lower concentration of vascular free cortisol makes it clear that most of the vascular cortisol is in bound states. The curve representing extravascular free cortisol concentration, X_{Fe}, shows that the extravascular volume contains the most cortisol because the extravascular volume is much larger (˜40×) than vascular volume. These volumes are different for each subject.

FIGS. 3B-3C show the same data on different scales. The curves in FIG. 3B represent the time from 0 to 7 minutes. There is only one single data point in this time range, at zero minutes. Thus, the curves presented at this scale are predictive and informative in that they lead smoothly into the time range for the remaining measurements. FIG. 3B shows a near instantaneous rise in concentrations in the model. This rise, in fact, would take some time in the human system. FIG. 3B shows that the initial administration of cortisol shows up primarily as very high concentrations of albumin-bound and free cortisol.

FIG. 3C shows the same data as FIG. 3A with an expanded timescale compared with FIG. 3A. In FIG. 3C, the curves represent the concentration of free cortisol in the vascular volume and in the extravascular volume. In FIG. 3C, the curves representing concentration of free cortisol in the vascular volume and in the extravascular volume cross at approximately eight minutes. Prior to this crossing, the difference in concentration of free cortisol drives diffusion from the vascular into the extravascular volume. At the crossing point, there is no net diffusion and for times greater than the crossing point the concentration of free cortisol in the extravascular volume exceeds that of the vascular volume. This indicates that upon initial administration of exogenous cortisol the majority of the cortisol appears in the vascular volume. This is reasonable considering that the administration of the cortisol is into the vascular volume. For a time period following the administration, there is a net diffusion of free cortisol from the vascular volume into the extravascular volume. Once the free vascular concentration drops below the extravascular, by virtue of this diffusion and elimination from vascular, the diffusion then changes direction and results in a net diffusion from the extravascular into the vascular volume. The time of vascular mixing may preclude such short-time measurements. The multi-compartment model accounts for all measured data including the point at zero, as well as at 10 minutes and greater.

TABLE II

| | α | k | V_{f} |
|---|---|---|---|
| ID | s⁻¹ | (molar*s)⁻¹ | (per unit) |
| 3 | 0.0144 | 0.0205 | 0.98 |
| 4 | 0.0163 | 0.0291 | 0.98 |
| 5 | 0.0108 | 0.0268 | 1.0 |
| 8 | 0.0152 | 0.0162 | 0.99 |
| 9 | 0.0131 | 0.0206 | 0.98 |
| 10 | 0.0121 | 0.0183 | 0.99 |

Table II shows the solutions for the three model unknowns for all subjects. The solutions listed in Table II are not identical as each subject was different in size, weight, gender, age and so on. Thus, it is to be expected to observe certain variability between subjects. The elimination rate constant, α, refers to the clearance of free cortisol from the vascular volume by renal clearance, hepatic clearance, and all other mechanisms by which free cortisol leaves the system from the vascular volume. It is understood that the bound fractions of cortisol do not clear directly but only by first disassociation to become free and then to clearance. This has been treated differently and not physiologically in the past in which clearance was taken to track the total vascular cortisol which is the sum of bound and unbound.

It is important to note that the extravascular volume is different. The model assumes that free cortisol enters by diffusion from the vascular system and resides there in a state, the details of which are not included in the model. Free cortisol does not clear directly from the extravascular volume but may diffuse into the vascular volume within which it may bind to a protein or clear from the system.

TABLE III

| | α | k | V_{fr} |
|---|---|---|---|
| X_{TotCBG} | 0.067 | 0.266 | 0.311 |
| X_{TotA} | 0.470 | 0.219 | 0.0364 |
| κ₁^{C} | 0.0018 | 0.325 | 0.318 |
| κ₋₁^{C} | 0.0995 | 0.213 | 0.315 |
| κ₁^{A} | 0.5188 | 0.477 | 0.212 |
| κ₋₁^{A} | 0.0128 | 0.0885 | 0.139 |

Table III shows the sensitivities of the solutions to the model parameters which were computed as the numerical derivatives of each solved parameter with respect to the row parameter. Table III shows the percent change in solution for a percent change in the associated model parameter. A value of 1 indicates that a 1% change in model parameter gives rise to a 1% change in solution. Table III shows that most of the sensitivities are below unity suggesting that solutions are not overly sensitive to parameter inaccuracies.

The sensitivities of all subject solutions were not identical since each had unique CBG concentrations and measurements. The largest sensitivity is 0.502 corresponding to just over a 0.5% error in α arising from a 1% error in measured X_{TotA}. The model sensitivities may be computed for any subject, and thus, sensitivities can be detected and examined. Considering the Taylor series expansion of the sensitivity function, the worst-case cumulative sensitivity is well below unity.

TABLE IV

| | α | k | V_{fr} |
|---|---|---|---|
| ID | s⁻¹ | (molar*s)⁻¹ | (per unit) |
| 1 | 0.0151 | 0.0210 | 0.99 |
| 2 | 0.0104 | 0.0233 | 1.0 |

Table IV shows the solutions for two CBG deficient subjects for which the model fit the measured data simply by using the measured CBG value and making a simple adjustment to κ₁^{C}, lowering it by a factor of 6 while all other parameters are derived similarly to the normal subjects discussed above.

FIG. 3D shows a graph of the concentration of CBG-bound cortisol for a CBG deficient subject. In FIG. 3D, the graph shows that the concentration of CBG-bound cortisol for a CBG deficient subject is much lower than in the case of the normal subject shown in FIG. 3A as expected. The model for total vascular cortisol (upper line) reasonably matches the measurements (encircled dots), which can be seen in FIG. 3D.

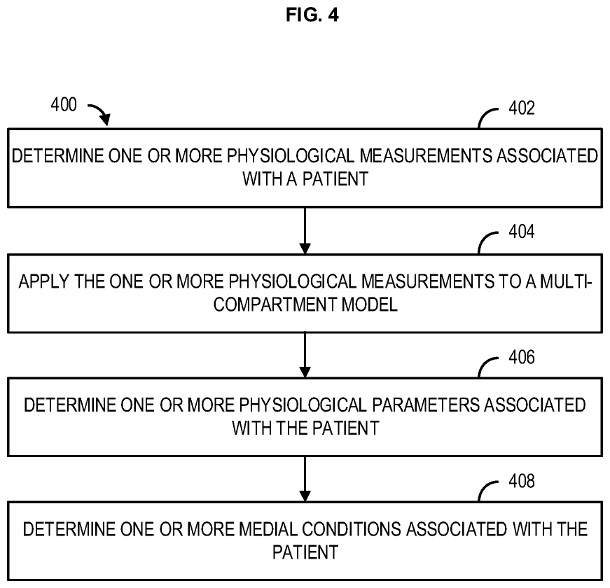

FIG. 4 shows an example method 400 for determining one or more physiological parameters associated with a patient based on one or more physiological measurements associated with the patient. Method 400 may be implemented by device 101, electronic device 102, server 106, or any combination thereof. At step 402, one or more physiological measurements associated with the patient may be determined. The one or more physiological measurements may comprise one or more of a time series of blood cortisol concentrations, plasma concentrations associated with corticosteroid binding globulin (CBG) and cortisol, a total CBG concentration, a total albumin concentration, a blood volume, a plasma volume, vascular volume, or extravascular volume.

At step 404, the one or more physiological measurements may be applied to a multi-compartment model. The multi-compartment model may represent a bidirectional flux by diffusion of a hormone between a vascular compartment and an extravascular compartment of the patient. The hormone may comprise one or more of free cortisol, testosterone, aldosterone, vitamin D, or a peptide hormone. The multi-compartment model may comprise four non-linear differential equations. The four non-linear differential equations may represent time-varying concentrations of plasma free cortisol in a vascular volume, CBG-bound cortisol in a vascular volume, albumin-bound cortisol in a vascular volume, and free cortisol in an extravascular volume.

At step 406, one or more physiological parameters associated with the patient may be determined based on the application of the one or more physiological measurements to the multi-compartment model. For example, the multi-compartment model may be solved, based on the one or more physiological measurements, for the one or more physiological parameters. The one or more physiological parameters may comprise one or more of free cortisol elimination rate (α), extravascular volume fraction (V_{f}), or a permeability constant related to diffusion (k).

At step 408, one or more medical conditions may be determined based on the one or more physiological parameters. For example, one or more physiological attributes associated with the patient maybe determined, or predicted, based on the one or more physiological parameters. The one or more medical conditions may be determined based on the one or more physiological attributes. The one or more physiological attributes may comprise time-varying concentrations of one or more of plasma free cortisol in a vascular volume, CBG-bound cortisol in a vascular volume, albumin-bound cortisol in a vascular volume, and free cortisol in an extravascular volume. The one or more medical conditions associated with the patient may comprise one or more of an adrenocortical disorder, an adrenal insufficiency, autonomous cortisol secretion, and a critical illness associated with corticosteroid insufficiency. In an example, a treatment of the one or more medical conditions may be administered to the patient.

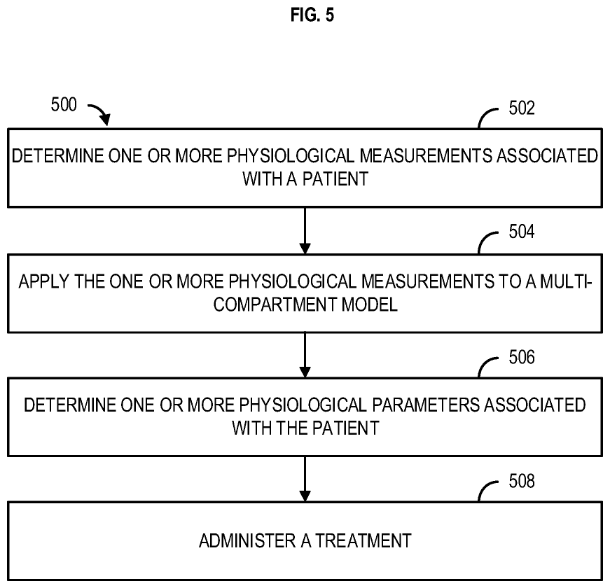

FIG. 5 shows an example method 500 for determining one or more physiological parameters associated with a patient based on one or more physiological measurements associated with the patient. Method 500 may be implemented by device 101, electronic device 102, server 106, or any combination thereof. At step 502, one or more physiological measurements associated with the patient may be determined. The one or more physiological measurements may comprise one or more of a time series of blood cortisol concentrations, plasma concentrations associated with corticosteroid binding globulin (CBG) and cortisol, a total CBG concentration, a total albumin concentration, a blood volume, a plasma volume, vascular volume, or extravascular volume.

At step 504, the one or more physiological measurements may be applied to a multi-compartment model. The multi-compartment model may represent a bidirectional flux by diffusion of a hormone between a vascular compartment and an extravascular compartment of the patient. The hormone may comprise one or more of free cortisol, testosterone, aldosterone, vitamin D, or a peptide hormone. The multi-compartment model may comprise four non-linear differential equations. The four non-linear differential equations may represent time-varying concentrations of plasma free cortisol in a vascular volume, CBG-bound cortisol in a vascular volume, albumin-bound cortisol in a vascular volume, and free cortisol in an extravascular volume.

At step 506, one or more physiological parameters associated with the patient may be determined based on the application of the one or more physiological measurements to the multi-compartment model. For example, the multi-compartment model may be solved, based on the one or more physiological measurements, for the one or more physiological parameters. The one or more physiological parameters may comprise one or more of free cortisol elimination rate (α), extravascular volume fraction (V_{f}), or a permeability constant related to diffusion (k).

At step 508, a treatment may be administered to the patient based on the one or more physiological parameters. As an example, one or more medical conditions may be determined based on the one or more physiological parameters associated with the patient. The treatment may be administered to the patient based on the one or more medical conditions. The one or more medical conditions associated with the patient may comprise one or more of an adrenocortical disorder, an adrenal insufficiency, autonomous cortisol secretion, and a critical illness associated with corticosteroid insufficiency. As a further example, one or more physiological attributes associated with the patient may be determined, or predicted, based on the one or more physiological parameters. The one or more medical conditions may be determined based on the one or more physiological attributes. The one or more physiological attributes may comprise time-varying concentrations of one or more of plasma free cortisol in a vascular volume, CBG-bound cortisol in a vascular volume, albumin-bound cortisol in a vascular volume, and free cortisol in an extravascular volume. As a further example, a treatment of the one or more medical conditions may be adjusted based on the one or more physiological parameters.

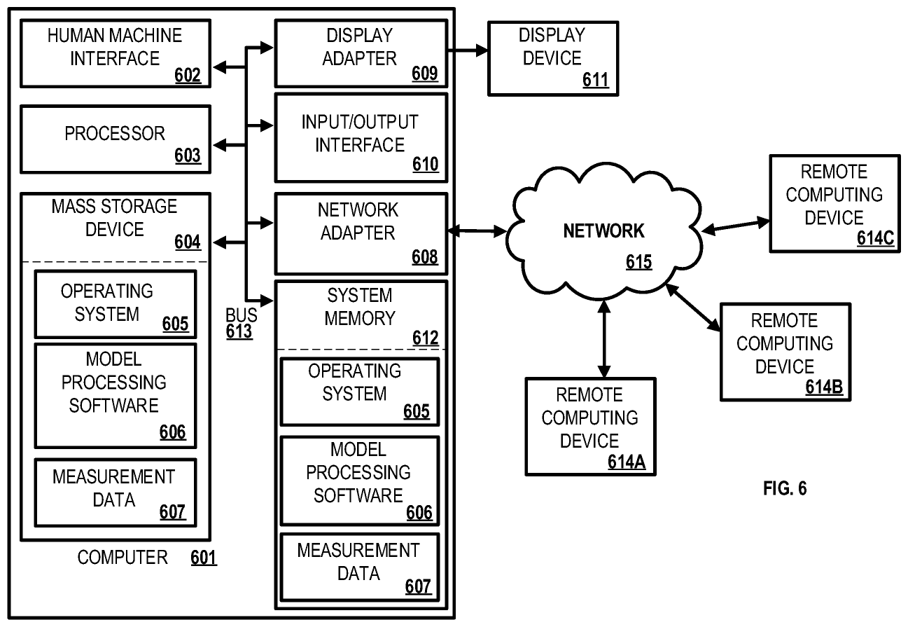

FIG. 6 a block diagram of another example computing device. In an exemplary aspect, the methods and systems can be implemented on a computer 601 as illustrated in FIG. 6 and described below. By way of example, the device 101, the electronic device 102, and the server 106 of FIG. 1 can be a computer 601 as illustrated in FIG. 6. Similarly, the methods and systems disclosed can utilize one or more computers to perform one or more functions in one or more locations. FIG. 6 is a block diagram illustrating an exemplary operating environment 600 for performing the disclosed methods. This exemplary operating environment 600 is only an example of an operating environment and is not intended to suggest any limitation as to the scope of use or functionality of operating environment architecture. Neither should the operating environment 600 be interpreted as having any dependency or requirement relating to any one or combination of components illustrated in the exemplary operating environment 600.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media including memory storage devices.

Further, one skilled in the art will appreciate that the systems and methods disclosed herein can be implemented via a general-purpose computing device in the form of a computer 601. The computer 601 can comprise one or more components, such as one or more processors 603, a system memory 612, and a bus 613 that couples various components of the computer 601 including the one or more processors 603 to the system memory 612. In the case of multiple processors 603, the system can utilize parallel computing.

The bus 613 can comprise one or more of several possible types of bus structures, such as a memory bus, memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 613, and all buses specified in this description can also be implemented over a wired or wireless network connection and one or more of the components of the computer 601, such as the one or more processors 603, a mass storage device 604, an operating system 605, model processing software 606, measurement data 607, a network adapter 608, system memory 612, an Input/Output Interface 610, a display adapter 609, a display device 611, and a human machine interface 602, can be contained within one or more remote computing devices 614A-614C at physically separate locations, connected through buses of this form, in effect implementing a fully distributed system.

The computer 601 typically comprises a variety of computer readable media. Exemplary readable media can be any available media that is accessible by the computer 601 and comprises, for example and not meant to be limiting, both volatile and non-volatile media, removable and non-removable media. The system memory 612 can comprise computer readable media in the form of volatile memory, such as random access memory (RAM), and/or non-volatile memory, such as read only memory (ROM). The system memory 612 typically can comprise data such as measurement data 607 and/or program modules such as operating system 605 and interference processing software 606 that are accessible to and/or are operated on by the one or more processors 603.

In another aspect, the computer 601 can also comprise other removable/non-removable, volatile/non-volatile computer storage media. By way of example, the computer 601 can comprise a mass storage device 604 which can offer non-volatile storage of computer code, computer readable instructions, data structures, program modules, and other data for the computer 601. For example, a mass storage device 604 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 604, including by way of example, an operating system 605 and model processing software 606. One or more of the operating system 605 and model processing software 606 (or some combination thereof) can comprise elements of the programming and the model processing software 606. Measurement data 607 can also be stored on the mass storage device 604. Measurement data 607 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple locations within the network 615.

In another aspect, the user can enter commands and information into the computer 601 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices can be connected to the one or more processors 603 via a human machine interface 602 that is coupled to the bus 613, but can be connected by other interface and bus structures, such as a parallel port, game port, an IEEE 1394 Port (also known as a Firewire port), a serial port, network adapter 608, and/or a universal serial bus (USB).

In yet another aspect, a display device 611 can also be connected to the bus 613 via an interface, such as a display adapter 609. It is contemplated that the computer 601 can have more than one display adapter 609 and the computer 601 can have more than one display device 611. For example, a display device 611 can be a monitor, an LCD (Liquid Crystal Display), light emitting diode (LED) display, television, smart lens, smart glass, and/or a projector. In addition to the display device 611, other output peripheral devices can comprise components such as speakers (not shown) and a printer (not shown) which can be connected to the computer 601 via Input/Output Interface 610. Any step and/or result of the methods can be output in any form to an output device. Such output can be any form of visual representation, including, but not limited to, textual, graphical, animation, audio, tactile, and the like. The display 611 and computer 601 can be part of one device, or separate devices.

The computer 601 can operate in a networked environment using logical connections to one or more remote computing devices 614A, 614B, and 614C. By way of example, a remote computing device 614A-614C can be a personal computer, a computing station (e.g., a workstation), a portable computer (e.g., a laptop, a mobile phone, a tablet device), a smart device (e.g., a smartphone, a smart watch, an activity tracker, a smart apparel, a smart accessory), a security and/or monitoring device, a server, a router, a network computer, a peer device, an edge device or other common network node, and so on. Logical connections between the computer 601 and a remote computing device 614A-614C can be made via a network 615, such as a local area network (LAN) and/or a general wide area network (WAN). Such network connections can be through a network adapter 608. A network adapter 608 can be implemented in both wired and wireless environments. Such networking environments are conventional and commonplace in dwellings, offices, enterprise-wide computer networks, intranets, and the Internet.

For purposes of illustration, application programs and other executable program components such as the operating system 605 are illustrated herein as discrete blocks, although it is recognized that such programs and components can reside at various times in different storage components of the computer 601, and are executed by the one or more processors 603 of the computer 601. An implementation of model processing software 606 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g., a genetic algorithms), swarm intelligence (e.g., an ant algorithms), and hybrid intelligent systems (e.g., expert inference rules generated through a neural network or production rules from statistical learning).

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
determining one or more physiological measurements associated with a patient;
applying the one or more physiological measurements to a multi-compartment model, wherein the multi-compartment model represents a bidirectional flux by diffusion of a hormone between a vascular compartment and an extravascular compartment of the patient;
determining, based on the application of the one or more physiological measurements to the multi-compartment model, one or more physiological parameters associated with the patient; and
determining, based on the one or more physiological parameters, one or more medical conditions associated with the patient.

2. The method of claim 1, The method of claim 1, wherein the one or more physiological measurements comprise one or more of a time series of blood cortisol concentrations, plasma concentrations associated with corticosteroid binding globulin (CBG) and cortisol, a total CBG concentration, a total albumin concentration, a blood volume, a plasma volume, vascular volume, or extravascular volume.

3. The method of claim 1, wherein the multi-compartment model comprises four non-linear differential equations, and wherein the four non-linear differential equations represent time-varying concentrations of plasma free cortisol in a vascular volume, CBG-bound cortisol in a vascular volume, albumin-bound cortisol in a vascular volume, and free cortisol in an extravascular volume.

4. The method of claim 1, wherein the hormone comprises one or more of free cortisol, testosterone, aldosterone, vitamin D, or a peptide hormone.

5. The method of claim 1, wherein the one or more physiological parameters comprise one or more of free cortisol elimination rate (α), extravascular volume fraction (V_{f}), or a permeability constant related to diffusion (k).

6. The method of claim 1, wherein determining, based on the one or more physiological parameters, the one or more medical conditions associated with the patient comprises:
determining, based on the one or more physiological parameters, one or more physiological attributes associated with the patient; and
determining, based on the one or more physiological attributes associated with the patient, the one or more medical conditions associated with the patient.

7. The method of claim 6, wherein the one or more physiological attributes comprise time-varying concentrations of plasma free cortisol in a vascular volume, CBG-bound cortisol in a vascular volume, albumin-bound cortisol in a vascular volume, and free cortisol in an extravascular volume.

8. The method of claim 1, wherein determining, based on the one or more physiological parameters, the one or more medical conditions associated with the patient comprises:
determining, based on the one or more physiological parameters, an appearance and elimination rate of the hormone in the patient; and
determining, based on the appearance and elimination rate of the hormone in the patient, the one or more medical conditions associated with the patient.

9. The method of claim 1, wherein the one or more medical conditions associated with the patient comprise one or more of an adrenocortical disorder, an adrenal insufficiency, autonomous cortisol secretion, and a critical illness associated with corticosteroid insufficiency.

10. The method of claim 1, further comprising causing a treatment of the one or more medical conditions associated with the patient.

11. A method comprising:
determining one or more physiological measurements associated with a patient;
applying the one or more physiological measurements to a multi-compartment model, wherein the multi-compartment model represents a bidirectional flux by diffusion of a hormone between a vascular compartment and an extravascular compartment of the patient;
determining, based on the application of the one or more physiological measurements to the multi-compartment model, one or more physiological parameters associated with the patient; and
administering, based on the one or more physiological parameters, a treatment associated with one or more medical conditions associated with the patient.

12. The method of claim 11, wherein the one or more physiological measurements comprise one or more of a time series of blood cortisol concentrations, plasma concentrations associated with corticosteroid binding globulin (CBG) and cortisol, a total CBG concentration, a total albumin concentration, a blood volume, a plasma volume, vascular volume, or extravascular volume.

13. The method of claim 11, wherein the multi-compartment model comprises four non-linear differential equations, and wherein the four non-linear differential equations represent time-varying concentrations of plasma free cortisol in a vascular volume, CBG-bound cortisol in a vascular volume, albumin-bound cortisol in a vascular volume, and free cortisol in an extravascular volume.

14. The method of claim 11, wherein the hormone comprises one or more of free cortisol, testosterone, aldosterone, vitamin D, or a peptide hormone.

15. The method of claim 11, wherein the one or more physiological parameters comprise one or more of free cortisol elimination rate (α), extravascular volume fraction (V_{f}), or a permeability constant related to diffusion (k).

16. The method of claim 11, wherein administering, based on the one or more physiological parameters, the treatment associated with the one or more medical conditions associated with the patient comprises:
determining, based on the one or more physiological parameters, one or more physiological attributes associated with the patient; and
administering, based on the one or more physiological attributes associated with the patient, the treatment associated with the one or more medical conditions associated with the patient.

17. The method of claim 16, wherein the one or more physiological attributes comprise time-varying concentrations of one or more of plasma free cortisol in a vascular volume, CBG-bound cortisol in a vascular volume, albumin-bound cortisol in a vascular volume, and free cortisol in an extravascular volume.

18. The method of claim 11, wherein administering, based on the one or more physiological parameters, the treatment associated with the one or more medical conditions associated with the patient comprises:
determining, based on the one or more physiological parameters, an appearance and elimination rate of the hormone in the patient; and
administering, based on the appearance and elimination rate of the hormone in the patient, the treatment associated with the one or more medical conditions associated with the patient.

19. The method of claim 11, wherein the one or more medical conditions associated with the patient comprise one or more of an adrenocortical disorder, an adrenal insufficiency, autonomous cortisol secretion, and a critical illness associated with corticosteroid insufficiency.

20. The method of claim 11, wherein administering, based on the one or more physiological parameters, the treatment of the one or more medical conditions associated with the patient comprises causing, based on the one or more physiological parameters, an adjustment of a treatment associated with the one or more medical conditions associated with the patient.
